(19) United States
(12) Patent Application Publication
(10) Pub. No.: US 2014/0271513 A1
(43) Pub. Date: Sep. 18, 2014

(54) FOOT ODOR REDUCTION COMPOSITION AND SYSTEM AND METHOD OF USE OF SAME

(71) Applicants: Makule Yesim Basar, Nyack, NY (US); Guher Gursel, Istanbul (TR)

(72) Inventors: Makule Yesim Basar, Nyack, NY (US); Guher Gursel, Istanbul (TR)

(21) Appl. No.: 13/835,854

(22) Filed: Mar. 15, 2013

(51) Int. Cl. A61K 8/34; A61K 8/41; A61K 8/49; A61K 8/19; A61Q 15/00; A61K 8/42

(52) U.S. Cl. CPC A61K 8/34; A61Q 15/00; A61K 8/42; A61K 8/498; A61K 8/19; A61K 8/41
USPC 424/65

(57) ABSTRACT

A foot odor reduction composition comprises ethanol, Vitamin B5, Vitamin E, and an effective amount of a mixture comprising an emulsifier, an antiseptic, and an ion holder for the application of foot odor.

### TECHNICAL FIELD OF THE INVENTION

The present invention relates to foot odor reduction compositions and systems and methods of use of the same.

### BACKGROUND

Body odor is formed when fresh perspiration, which is odorless at its outset, is decomposed by microorganisms. This process takes place at a number of locations in the body, including the foot and toe areas. A number of microorganisms are involved in the decomposition process and each has different activity leading to body odor of different strength and unpleasantness. An exceptional odor-producing microorganism may include aerobic diphtheroids, primarily Corynebacterium species, and coagulase negative cocci such as Micrococcaceae.

A broad array of topical products and personal hygiene products are available for application to human skin to counteract body odors, particularly those body odors resulting from and associated with perspiration. These products include deodorants, antiperspirants, foot and body powders, and body sprays.

However, these products typically require multiple applications to the affected area over a short period of time, for example, less-than-weekly applications to the odor source, in order to remain effective for an extended period of time. In addition, many of these products inhibit natural perspiration necessary for maintaining healthy skin.

Therefore, there remains a need for a topical product that can counteract body odor over an extended period of time through the use of limited applications that avoid interference with natural perspiration associated with healthy skin.

### SUMMARY OF THE INVENTION

A composition composed of ethanol, Vitamin B5, Vitamin E, and an effective amount of a mixture comprising an emulsifier, an antiseptic, and an ion holder may be used for foot odor reduction. The composition also may be composed of a desiccant, a barrier, a moisturizer, a solvent, at least one softening agent, a dampening agent, an anti-irritant, and at least one preservative, and is useable to substantially reduce foot odor at a target site following administration.

A foot odor reduction composition is composed of a desiccant, such as talcum, a barrier, such as zinc oxide, a moisturizer, such as lanolin, a solvent, such as water, a softening agent, such as natural glycerin or petrolatum jelly, a dampening agent, such as liquid paraffin, an anti-irritant, such as allantoin, and a preservative, such as DMDM hydantoin, methylchloroisothiazolinone, or methylisothyazolinone.

A foot odor reduction composition composed of an effective amount of ethanol and Vitamin B5, and an effective amount of a mixture of an emulsifier, an antiseptic, a solvent, an fragrance and an ion holder, after about 8 hours from the time of application, substantially reduces heavy to medium foot odor from a single administration to a target site.

A foot odor reduction composition substantially reduces heavy to medium foot odor for a period of up to about 90 days following about 8 hours after administration of the composition at the target site.

### BRIEF DESCRIPTION OF THE DRAWINGS

The present disclosure will become more fully understood from the detailed description and the accompanying, interrelated embodiments.

FIG. 1 illustrates an exemplary method of fabrication of an exemplary foot odor reduction composition.

In the drawings like characters of reference indicate corresponding parts in the different figures. The drawings are non-limiting examples of the disclosed embodiments of the present invention.

### DETAILED DESCRIPTION

All parts, percentages and ratios used herein are expressed by weight unless otherwise specified. Unless expressly stated, all compounds identified shall also include any isomers of the recited compounds and any of their analogues or homologues known to those skilled in the art.

An exemplary foot odor reduction composition may comprise a desiccant, such as talcum, a barrier, such as zinc oxide, a moisturizer, such as lanolin, a solvent such as water, a softening agent, such as natural glycerin or petrolatum jelly, a dampening agent, such as liquid paraffin, an anti-irritant, such as allantoin, one or more fragrances, and one or more preservatives, such as 1,3-Bis-(hydroxymethyl)-5,5-dimethylimidazolidine-2,4-dione (DMDM) hydantoin, methyl-chloro-isothiazolinone, or methyl-isothyazolinone.

An exemplary fragrance may be one or more of the following, alone or in combination, amyl-cinnamal, amyl-cinnamyl-alcohol, anisyl-alcohol, benzyl alcohol, benzyl benzoate, benzyl cinnamate, benzyl salicylate, cinnamal, cinnamyl alcohol, citral, citronellol, coumarin, eugenol, farnesol, geraniol, hexyl cinnamal, hydroxycitronellal, isoeugenol, and lilial, limonene,

An exemplary foot odor reduction composition may also comprise vitamins such as Vitamin B5 and Vitamin E. Those skilled in the art may recognize that stereoisomers of such vitamins and their metabolites or conjugated salts, such as calcium pantothenate or the α-, β-, γ-, and δ-tocopheryl acetates, may also be suitable for use in an exemplary foot odor reduction composition. Preferably, the foot odor reduction composition includes dexpanthenol and tocopheryl acetate.

An exemplary foot odor reduction composition may additionally comprise active ingredients dexpanthenol and ethanol and an active mixture of a solvent, such as monopropylene glycol, an emulsifier, such as polysorbate-20, an fragrance, an antiseptic, such as boric acid, and an ion holder, such as ethylenediaminetetraacetic acid (EDTA).

In an exemplary embodiment, a foot odor reduction composition may comprise about 4.00% to about 55.00% desiccant, about 4.00% to about 15.00% barrier material, about 1.00% to about 10.00% moisturizer, about 3.00% to about 15.00% solvent, about 1.00% to about 6.00% dexpanthenol, about 2.00% to about 28.00% softening agent, about 2.00% to about 8.00% ethanol, about 2.00% to about 13.00% dampening agent, about 0.010% to about 2.00% anti-irritant, about 0.010% to about 0.700% preservatives, about 0.10% to about 5.00% of a fragrance, and a mixture of monopropylene glycol, polysorbate-20, malodor, boric acid, and EDTA.

An exemplary malodor may include a one or more exemplary fragrances disclosed herein.

Alternatively, an exemplary foot odor reduction composition may comprise active ingredients of about 1.00% to about 6.00% dexpanthenol, about 2.00% to about 8.00% ethanol, about 1.00% to about 7.00% monopropylene glycol, about 0.50% to about 6.00% polysorbate-20, about 0.50% to about 6.00% malodor, about 0.20% to about 5.00% boric acid, and about 0.001% to about 0.500% EDTA.

In an exemplary embodiment, a foot odor reduction composition may comprise about 4.00% to about 55.00% talcum, about 4.00% to about 15.00% zinc oxide, about 1.00% to about 10.00% lanolin, about 3.00% to about 15.00% water, about 1.00% to about 6.00% dexpanthenol, about 2.00% to about 10.00% natural glycerin, about 0.10% to about 3.00% tocopheryl acetate, about 2.00% to about 8.00% ethanol, about 2.00% to about 13.00% liquid paraffin, about 9.00% to about 18.00% petrolatum jelly, about 0.010% to about 2.00% allantoin, about 0.010% to about 0.200% DMDM hydantoin, about 0.010% to about 0.200% methyl-chloro-isothiazolinone, about 0.010% to about 0.300% methylisothyazolinone, about 0.10% to about 5.00% of a fragrance, and a mixture of about 1.00% to about 7.00% monopropylene glycol, about 0.50% to about 6.00% polysorbate-20, about 0.50% to about 6.00% malodor, about 0.20% to about 5.00% boric acid, and about 0.001% to about 0.500% EDTA.

According to an exemplary embodiment of a foot odor reduction composition, the foot odor reduction composition may be a cream, gel, or an emulsion.

A preferred foot odor reduction composition may comprise a homogenous emulsion of talcum, zinc oxide, lanolin, water, tocopheryl acetate, natural glycerin, petrolatum jelly, liquid paraffin, allantoin, a fragrance, DMDM hydantoin, methylchloroisothiazolinone, methylisothyazolinone, and active ingredients dexpanthenol, ethanol, and a mixture of monopropylene glycol, polysorbate-20, fragrance, boric acid, and EDTA. In an exemplary homogenous foot odor reduction composition, a 2% emulsion of a homogenous foot odor reduction composition may comprise about 66% non-active ingredients.

A preferred foot odor reduction composition may be a highly viscous cream comprising about 10% to about 50% talcum, about 12% to about 20% zinc oxide, about 5% to about 12% lanolin, about 3% to about 10% deionized water, about 0.1% tocopheryl acetate, about 2% to about 8% natural glycerin, about 4% to about 10% petrolatum jelly, about 4% to about 9% liquid paraffin, about 0.2% to about 5.0% allantoin, about 5% of a fragrance, about 1.6% to about 8.0% dexpanthenol, about 3% to about 9% ethanol, and a mixture, which includes monopropylene glycol, polysorbate-20, a fragrance, boric acid, and EDTA, of about 5% to about 17%.

The viscosity of the foot odor reduction composition changes in relation to the level of the desiccant, the moisturizer, the barrier, and the softening agent. In a preferred embodiment, the viscosity of an exemplary foot odor reduction composition may vary by the weight percentages of the following ingredients: talcum, lanolin, and petrolatum jelly. In another preferred embodiment, the viscosity of an exemplary foot odor reduction composition may vary by the weight percentage of the barrier constituent, such as zinc oxide.

An exemplary foot odor reduction composition in the form of a high viscosity cream (approximately 40.00 cP) may be about 50% by weight of a mixture containing talcum, lanolin and petrolatum jelly. Preferably, a high viscosity foot odor reduction composition of about 20.00 cP to about 40.00 cP contains about 10% to about 50% talcum, about 5% to about 12% lanolin and about 4% to about 10% petrolatum jelly.

In yet another exemplary embodiment, a foot odor reduction composition may contain an effective amount of an emulsifier, such as polysorbate-20, to increase melting temperatures of the foot odor reduction composition and prolong product shelf life. In a preferred embodiment, an exemplary foot odor reduction composition containing an effective amount of polysorbate-20 may have a shelf-life of 36 months (when sealed), an open container life of 12 months, and temperature stability of up to approximately 115° Fahrenheit when exposed to such temperature over a course of about 72 hours.

According to an exemplary embodiment, a foot odor reduction composition may be applied to the target site on the foot and used in conjunction with a cover apparatus or cover for the foot, such as, for example, a sock or bandages, comprised of about 85% cotton and about 15% polymer of the exemplary formula:

[chemical structure]

where x=about 40.

An exemplary polymer may be Lycra or other polyurethane derivatives and/or stretch fabrics. An exemplary cover used in conjunction with an exemplary foot odor reduction composition may also be antibacterial. Additionally, an exemplary cover may increase the absorption of an exemplary foot odor reduction composition through the skin of the user thereby increasing the effectiveness of the foot odor reduction composition.

In an exemplary method of use of an exemplary foot odor reduction composition, a target area of the foot, such as the soles, heels, or toes, is washed for approximately 10 minutes in warm water or may be pre-treated to moisten and/or exfoliate the foot skin using exfoliating materials known to those skilled in the art. Accordingly, the target area of the foot may have substantially all dead skin layers removed and may be prepared for application of the exemplary foot odor reduction composition. In an exemplary embodiment, the exemplary foot odor reduction composition is applied to the target area of the foot and covered by a sock comprised of cotton and Lycra for approximately 8-hours.

In an exemplary method of use of an exemplary foot odor reduction composition and exemplary cover, the foot odor reduction composition may have a dermal absorption rate of approximately 2-3 hours from the time of application to the dermal region and covering by an exemplary cover.

According to the exemplary method of use of an exemplary foot odor reduction composition, substantially complete removal of foot odor was achieved within about 2 to about 6 hours from the time of removing the cover from the target area. Further, use of the exemplary method described herein achieves substantially complete removal of foot odor present at the target area of the foot for approximately three months from the time at which the cover is removed from the target area. According to the exemplary method of use, foot odor at a target area of the foot may be substantially completely removed following a single application of the exemplary foot odor reduction composition to the target area.

In order that the invention may be more readily understood, reference is made to the following examples which are intended to illustrate the results of using the exemplary foot odor reduction composition according to the methods described herein without serving as limitations on the foot odor reduction composition or its methods of use.

Subjects were characterized as having a medium- to heavy-foot odor by the distance from the subject's feet at which the foot odor would cease. The foot odors varied, but were described as having a foul smell, analogous to that of malt vinegar. These odor tests were conducted in a laboratory with minimal to no air movement to preclude the use of external forces to displace the odors from the subjects' feet to the detection area. Accordingly, a foul smell that persisted until about a 10-foot distance from the subject's bare feet would be characterized as an exemplary medium-level foot odor. A foul smell that persisted until about a 25-foot distance from the subject's bare feet would be characterized as an exemplary high-level foot odor. Each of the subjects was found to have foot odor detectable at a range of about 10 feet to about 25 feet from the subject's bare feet.

### Example 1

A 19-year old male subject identified as having heavy foot odor (in the range of about 15 to about 25 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Heavy foot odor was absent for 92 days from the date of application of the foot odor reduction composition.

### Example 2

A 36-year old male subject identified as having medium foot odor (in the range of about 10 to about 15 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Medium foot odor was absent for 94 days from the date of application of the foot odor reduction composition. After 94 days, the subject's foot odor was less than medium (in a range below about 10 feet).

### Example 3

A 39-year old female subject identified as having heavy foot odor (in the range of about 15 to about 25 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Heavy foot odor was absent for 89 days from the date of application of the foot odor reduction composition.

### Example 4

A 42-year old female subject identified as having medium foot odor (in the range of about 10 to about 15 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Medium foot odor was absent for 93 days from the date of application of the foot odor reduction composition.

### Example 5

A 27-year old female subject identified as having heavy foot odor (in the range of about 15 to about 25 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Heavy foot odor was absent for 89 days from the date of application of the foot odor reduction composition.

### Example 6

A 19-year old female subject identified as having heavy foot odor (in the range of about 15 to about 25 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Heavy foot odor was absent for 87 days from the date of application of the foot odor reduction composition. After 87 days, the subject's foot odor was no longer heavy, but medium (in the range of about 10 to about 15 feet).

### Example 7

A 32-year old male subject identified as having heavy foot odor (in the range of about 15 to about 25 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Heavy foot odor was absent for 93 days from the date of application of the foot odor reduction composition.

### Example 8

A 47-year old male subject identified as having medium foot odor (in the range of about 10 to about 15 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Medium foot odor was absent for 97 days from the date of application of the foot odor reduction composition.

### Example 9

A 58-year old male subject identified as having heavy foot odor (in the range of about 15 to about 25 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Heavy foot odor was absent for 91 days from the date of application of the foot odor reduction composition.

### Example 10

A 52-year old male subject identified as having medium foot odor (in the range of about 10 to about 15 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Medium foot odor was absent for 96 days from the date of application of the foot odor reduction composition.

### Example 11

A 28-year old male subject identified as having heavy foot odor (in the range of about 15 to about 25 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Heavy foot odor was absent for 89 days from the date of application of the foot odor reduction composition.

### Example 12

A 61-year old male subject identified as having medium foot odor (in the range of about 10 to about 15 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Medium foot odor was absent for 87 days from the date of application of the foot odor reduction composition.

### Example 13

A 38-year old male subject identified as having medium foot odor (in the range of about 10 to about 15 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Medium foot odor was absent for 93 days from the date of application of the foot odor reduction composition.

### Example 14

A 41-year old male subject identified as having a heavy foot odor (in the range of about 15 to about 25 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Heavy foot odor was absent for 88 days from the date of application of the foot odor reduction composition.

### Example 15

A 51-year old male subject identified as having heavy foot odor (in the range of about 15 to about 25 feet) received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Heavy foot odor was absent for 88 days from the date of application of the foot odor reduction composition.

### Example 16

A 58-year old female subject identified as having medium foot (in the range of about 10 to about 15 feet) odor received application with an exemplary foot odor reduction composition. The subject's feet were washed in warm water and soaked for approximately 10 minutes. Dead skin layers were removed from the subject's feet. The foot odor reduction composition was applied on the subject's foot skin. Following a single application of the foot odor reduction composition to the subject's feet, the subject wore about 85% cotton and about 15% Lycra socks for about 8 hours. The foot odor disappeared in the subject within 2-6 hours after removal of the cotton and Lycra socks. The subject was monitored monthly for recurrence of the foot odor using the same odor detection methodologies described. The subject did not use any other foot product other than the applied foot odor reduction composition. Medium foot odor was absent for 93 days from the date of application of the foot odor reduction composition.

During the testing identified in each of the disclosed examples 1 through 16, the foot odor reduction composition exemplified by the disclosures herein provided additional benefits, such as the moisturizing of the subjects' feet, removal of heel cracks, and a reported fresh scent.

FIG. 1 illustrates an exemplary method of manufacturing an exemplary foot odor reduction composition. According to the exemplary step 101, talcum, zinc oxide, water, glycerin are mixed. According to the exemplary step 102, approximately an effective amount of monopropylene glycol, polysorbate-20, malodor/fragrance, boric acid, and EDTA may be mixed in liquid form with the mixture of step 101. In step 103, the mixture of 102 may be mixed with lanolin, petroleum and liquid paraffin. In step 104, the mixture of 103 may be heated, preferably in a stainless steel apparatus, to approximately 185° Fahrenheit and in step 105, the mixture may be slowly mixed, preferably in liquid form, which may be achieved by a reduction in heating of the mixture, for example, a reduction in heating of the mixture to 68° Fahrenheit.

As illustrated in an exemplary step 106 of an exemplary method of manufacturing an exemplary foot odor reduction composition, a fragrance, DMDM hydantoin, methyl-chloro-isothiazolinone, and methyl-isothiazolinone are mixed, preferably in a liquid form. In an exemplary step 107, dexpanthenol, allantoin, and tocopheryl acetate are mixed together with the mixture of step 106 at approximately 104° Fahrenheit.

An exemplary step 108 may involve the mixture of the results of steps 105 and 107 following mixing of approximately 4 hours. An exemplary foot odor reduction composition may result from the mixture at step 109.

In accordance with previously-disclosed embodiments, an exemplary method of manufacture may be run iteratively via step 110 to achieve a desired viscosity by varying one or more of the desiccant, the moisturizer, the barrier, and/or the softening agent.

An exemplary foot odor reduction composition may significantly reduce organisms generally associated with foot odor, such as, eschericia coli, staphylococcus aureus, pseudomonas aeruginosa, aspergillus brasiliensis, and candida albicans.

It will be understood that the embodiments described herein are merely exemplary and that a person skilled in the art may make many variations and modifications without departing from the spirit and scope of the invention. All such variations and modifications are intended to be included within the scope of the invention as defined in the appended claims. Changes may be made, within the purview of the appended claims without departing from the scope and spirit of the system and method in their broader aspects. These disclosures should be understood to involve interrelationships between the various structures, materials, and embodiments, and it is understood that such interrelationships are not limited to the particulars disclosed.

I claim:

1. A foot odor reduction composition, comprising ethanol, Vitamin B5, Vitamin E, and an effective amount of a mixture comprising an emulsifier, an antiseptic, and an ion holder.

2. The composition of claim 1, wherein the emulsifier is polysorbate-20, the antiseptic is boric acid, and the ion holder is ethylenediaminetetraacetic acid.

3. The composition of claim 2, wherein the mixture further comprises a mixture solvent and an fragrance.

4. The composition of claim 3, wherein the mixture solvent is monopropylene glycol.

5. The composition of claim 4, wherein the mixture of polysorbate-20, boric acid, ethylenediaminetetraacetic acid, monopropylene glycol, and the fragrance is about 5 to about 17 weight by weight percentage.

6. The composition of claim 1, further comprising a desiccant, a barrier, a moisturizer, a solvent, a fragrance, at least one softening agent, a dampening agent, an anti-irritant, and at least one preservative.

7. The composition of claim 6, wherein the desiccant is talcum, the barrier is zinc oxide, the moisturizer is lanolin, the solvent is water, the softening agent is at least one of natural glycerin or petrolatum jelly, the dampening agent is liquid paraffin, the anti-irritant is allantoin, and the preservative is at least one of DMDM hydantoin, methylchloroisothiazolinone, or methylisothyazolinone.

8. The composition of claim 6, wherein the emulsifier is polysorbate-20, the antiseptic is boric acid, and the ion holder is ethylenediaminetetraacetic acid.

9. The composition of claim 6, further comprising an effective amount of a mixture of polysorbate-20, boric acid, ethylenediaminetetraacetic acid, monopropylene glycol, and an fragrance.

10. The composition of claim 6, wherein
the desiccant is about 4.00 to about 55.00 weight by weight percentage;
the barrier is about 4.00 to about 15.00 weight by weight percentage;
the moisturizer is about 1.00 to about 10.00 weight by weight percentage;
the fragrance is about 0.100 to about 5.00 weight by weight percentage;
the solvent is about 3.00 to about 15.00 weight by weight percentage;
the Vitamin B5 is about 1.00 to about 6.00 weight by weight percentage;
the at least one softening agent is about 2.00 to about 18.00 weight by weight percentage;
the Vitamin E is about 0.10 to about 3.00 weight by weight percentage;
the ethanol is about 2.00 to about 8.00 weight by weight percentage;
the dampener is about 2.00 to about 13.00 weight by weight percentage;
the anti-irritant is about 0.010 to about 2.00 weight by weight percentage;
the at least one preservative is about 0.010 to about 0.300 weight by weight percentage;
the emulsifier is about 0.50 to about 6.00 weight by weight percentage;
the antiseptic is about 0.20 to about 5.00 weight by weight percentage; and,
the ion holder is about 0.001 to about 0.500 weight by weight percentage.

11. The composition of claim 10, wherein a first of the at least one softening agent is about 2.00 to about 10.00 weight by weight percentage.

12. The composition of claim 11, wherein a second of the at least one softening agent is about 9.00 to about 18.00 weight by weight percentage.

13. The composition of claim 10, wherein a first of the at least one preservative is about 0.010 to about 0.200 weight by weight percentage.

14. The composition of claim 13, wherein a second of the at least one preservative is about 0.010 to about 0.300 weight by weight percentage.

15. The composition of claim 9, wherein
the desiccant is about 4.00 to about 55.00 weight by weight percentage;
the barrier is about 4.00 to about 15.00 weight by weight percentage;
the moisturizer is about 1.00 to about 10.00 weight by weight percentage;
the fragrance is about 0.100 to about 5.00 weight by weight percentage;
the solvent is about 3.00 to about 15.00 weight by weight percentage;
the Vitamin B5 is about 1.00 to about 6.00 weight by weight percentage;
the at least one softening agent is about 2.00 to about 18.00 weight by weight percentage;
the Vitamin E is about 0.10 to about 3.00 weight by weight percentage;
the ethanol is about 2.00 to about 8.00 weight by weight percentage;
the dampener is about 2.00 to about 13.00 weight by weight percentage;
the anti-irritant is about 0.010 to about 2.00 weight by weight percentage;
the at least one preservative is about 0.010 to about 0.300 weight by weight percentage;
the polysorbate-20 is about 0.50 to about 6.00 weight by weight percentage;
the boric acid is about 0.20 to about 5.00 weight by weight percentage;
the ethylenediaminetetraacetic acid is about 0.001 to about 0.500 weight by weight percentage;
the monopropylene glycol is about 1.00 to about 7.00 weight by weight percentage; and
the fragrance is about 0.50 to about 6.00 weight by weight percentage.

16. A foot odor reduction composition, comprising an effective amount of ethanol and Vitamin B5, and an effective amount of a mixture of an emulsifier, an antiseptic, a solvent, an fragrance and an ion holder, wherein the composition after about 8 hours substantially reduces heavy to medium foot odor from a single administration to a target site.

17. The foot odor reduction composition of claim 16, wherein the composition substantially reduces heavy to medium foot odor for a period of at least 7 days.

18. The foot odor reduction composition of claim 16, wherein the composition substantially reduces heavy to medium foot odor for a period of at least 28 days.

19. The foot odor reduction composition of claim 16, wherein the composition substantially reduces heavy to medium foot odor for a period of at least 56 days.

20. The foot odor reduction composition of claim 16, wherein the composition substantially reduces heavy to medium foot odor for a period of at least 90 days.
